(19) United States
(12) Patent Application Publication
(10) Pub. No.: US 2008/0152673 A1
(43) Pub. Date: Jun. 26, 2008

(54) Desiccated Product

(75) Inventor: Jeff Drew, Copthorne (GB)

Correspondence Address: CLARK & ELBING LLP, 101 FEDERAL STREET, BOSTON, MA, 02110, US

(73) Assignee: Stabilitech Ltd., London (GB)

(21) Appl. No.: 11/815,947

(22) PCT Filed: Feb. 9, 2006
(86) PCT No.: PCT/GB2006/000458

§ 371 Date: Aug. 9, 2007

(30) Foreign Application Priority Data
Feb. 9, 2005 (GB) 0502661.2

(51) Int. Cl. A61K 39/00; C12N 9/00; C12N 7/00; C07K 16/00; C12N 5/04; C12N 5/02; C12N 1/20; C12N 5/06; C07K 14/00; C07H 21/00

(52) U.S. Cl. 424/278.1; 435/183; 435/235.1; 530/387.1; 530/300; 530/350; 536/23.1; 435/325; 435/410; 435/243; 435/374; 435/260

(57) ABSTRACT

A desiccated or preserved product. The product comprises a sugar, a charged material and a sensitive biological component. The sugar forms an amorphous solid matrix.

### FIELD OF THE INVENTION

The present invention relates to a desiccated or preserved product and, more specifically, a desiccated product comprising a biological component. The invention also relates to a method of preserving a biological component, and to the use of a mixture comprising sugar for the preservation of a biological component.

### BACKGROUND

Intolerance of desiccation and thermal variation of many compounds, including biologically active molecules e.g. virus particles, is well documented e.g. heating a solution of adenovirus to 56° C. for 30 minutes is sufficient to eliminate infectious viruses. However a dry formulation which maintains the function of the compound for use, e.g. maintaining virus infectivity and vaccine efficacy, is desirable in many instances. Conferring desiccation tolerance and thermostability to viral particles, holds many potential applications, including extension of shelf life, ease of storage and transport with ease worldwide.

Biomimicry has been successfully applied in many instances to obtain novel applications from natural mechanisms. Desiccation tolerance has been observed in several biological settings other than plant seed maturation. So called “resurrection plants” (Selaginella and Myrothamnus), Tardigrade (Echiniscoides sigimunde), and brine shrimps (Artemia) are all capable of withstanding extended periods of anhydrobiosis. Although in these cases it has been suggested that the sugar trehalose, behaving as a water replacement molecule, is responsible for desiccation tolerance (Clegg 1986; Crowe et al 1987, 1992), it is sucrose which forms the most abundant sugar in higher order plant seeds and which has been postulated to perform the same function in this setting.

The relative proportions of desiccation protective saccharides found in seeds vary, with the non-reducing disaccharide e.g. sucrose, and oligosaccharides e.g. raffinose, stachyose, verbascose, melezitose, forming differing portions of the total dry weight of the seed. Many compounds are produced and laid down in the maturing seed which play a role in desiccation tolerance of the seed (including galactosyl cyclitols and late embryogenesis abundant proteins (LEAs)). The relative concentrations of these additional compounds also vary between seeds of different origins.

Compounds frequently observed to accumulate in developing seeds include disaccharides such as sucrose and trehalose, trisaccharides such as umbelliferose, along with oligosaccharides such as raffinose, stachyose, verbascose or melezitose and galactosyl cyclitols. Additionally, LEAs, which comprise a complex set of robust hydrophilic proteins (Galau et al 1986) accumulate in the latter stages of seed maturation and have been associated with acquisition of desiccation tolerance prior to maturation drying in orthodox seeds (Bewley and Oliver 1992; Kermode 1997).

The accumulation of non-reducing sugars, particularly those of the raffinose series (Koster and Leopold 1988; Leprince et a/1990; Blackman et a/1992) and/or galactosyl cyclitols (Horbowicz and Obendorf 1994; Obendorf 1997) has been implicated in desiccation tolerance.

During the process of seed maturation, sucrose, in the presence of oligosaccharides is prevented from crystallisation and has a role in the desiccation tolerance of some seeds and pollens (e.g. Leopold and Vertucci 1986; Crowe et al 1987, 1992; Hoekstra and van Roekel 1988; Hoekstra et a/1991).

Two explanations have been proposed for the protective effects afforded by the various compounds. The first, the so-called “water replacement hypothesis” (Clegg 1986; Crowe et al 1992) suggests that the compounds displace water from, amongst other things, membrane surfaces, hence maintaining the lipid bi-layer.

The second explanation involves aqueous phase vitrification which generates what is commonly termed the “glassy state” (Koster and Leopold 1988; Williams and Leopold 1989; Koster 1991; Leopold et a/1994; Obendorf 1997). This mechanism is based on the observation that upon loss of water, sucrose and associated oligosaccharides (or galactosyl cyclitols) form high viscosity, amorphous super saturated solutions. Even at extremely low temperatures, a glass phase does not freeze and can be melted into a liquid phase without cellular injury simply by the addition of water (Bruni 1989). Due to the high viscosity of the glass, compounds trapped within it are held in a form of “stasis”, where chemical reactions and degradative processes proceed at negligible rates (Koster 1994). It has been suggested that within the seed, the glasses themselves do not confer desiccation tolerance per se, but contribute to the stability of the seed components in the dry state (Leopold et al 1994). Glass formation is a common characteristic of desiccation tolerant tissues.

Other compounds are implicated in desiccation tolerance (and tolerance to thermal variation). An important class of compounds, the late embryogenesis abundant proteins (LEAs), are expressed to high levels in mature seeds, and disappear shortly after germination (Galau et al 1991), suggesting an important role in desiccation tolerance (Wolkers et al 1995). Many of these proteins have been isolated and many are hydrophilic and highly charged.

Notwithstanding these observations, prior art methods of preserving biological components still suffer from the problem that they are unable to preserve the biological components in a biologically active form for extended periods of time at higher temperatures (e.g. above 4° C.). This is particularly a problem for the transport of, for example, live vaccines in circumstances where refrigeration is not available.

For example, WO01/37656 discloses the preservation of Bovine Respiratory Syncytial Virus in a solution of 2:1 sucrose:methylα-d-glucopyranoside but the resulting product is stored at 4° C. under vacuum.

While WO2005/040398 does not relate to viruses, it discloses loading a disaccharide into mammalian nucleated cells but instructs that, once dried, the cells are preferably stored at 4° C. (i.e. under refrigeration) and under vacuum.

The present invention seeks to alleviate one or more of the above problems.

In the present invention, methods observed during seed maturation to withstand desiccation and thermal damage have been adapted to protect sensitive biological molecules similarly, by mixing certain biological compounds with the sugars and other compounds (or their functional equivalents e.g. histone proteins) implicated in protecting the integrity of seeds prior to desiccation (e.g. by lyophilisation). The resulting product is a highly stable, dry solid.

It has now been found that it is possible to adapt the protective mechanisms afforded to seeds and pollen during periods of desiccation to the purpose of preserving thermal or desiccation sensitive molecules, especially biological components (e.g. virus particles) during variations in storage conditions.

### SUMMARY OF THE INVENTION

This present invention involves conferring desiccation and thermal tolerance to materials which are normally desiccation or thermo sensitive using compositions which form a water-soluble vitreous (glass-like) matrix suitable for the purpose.

The invention uses biomimicry to adapt the protective methods used in the plant kingdom for seed and pollen desiccation and thermal stability to otherwise sensitive biological molecules e.g. virus particles and other compounds. This enables easier storage, transportation, production and administration e.g. for methods employing virus particles, viral vaccines and viral vectors.

One aspect of the invention requires mixing of certain sugars which aid desiccation tolerance in seeds (e.g. sucrose, trehalose, umbelliferose, raffinose, stachyose, verbascose, melezitose) with other compounds such as LEAs or proteins or peptides with similar physical characteristics (e.g. hydrophilicity or charge) isolated from other sources (e.g. mammalian cells or synthesised) with a desiccation or thermo sensitive substance in such a way that crystallisation is prevented when dried by methods known in the art, including lyophilisation, and desiccation or thermal tolerance is conferred upon the formerly sensitive substance.

In one embodiment of the invention, highly concentrated or saturated solutions of the sugars can be mixed with biological compounds in order to cause microscopic desiccation by osmosis of the compound to be protected prior to final drying by known methods e.g. lyophilisation.

Once mixed with the desiccation protecting medium, the samples can be dried to various residual moisture content e.g. 0.1 g H₂O g⁻¹ dry weight, to offer long term stability at greater than refrigeration temperatures e.g. within the range from about 4° C. to about 45° C. or above.

Addition of the compounds shown to be necessary for desiccation tolerance in seeds (e.g. LEAs) or proteins or peptides derived from natural sources (e.g. plant or mammalian origins (e.g. histone) or synthesised, with similar physical characteristics (e.g. charge or hydrophilicity)) further enhances the desiccation tolerance of the biological compound to be preserved.

According to one aspect of the present invention, there is provided a desiccated or preserved product comprising: a sugar; and a biological component.

According to another aspect of the present invention, there is provided the use of a sugar for the preservation of a biological component.

According to a further aspect of the present invention, there is provided a method of preserving a biological component comprising mixing the biological component with sugar.

Preferably, a charged material such as a protein is also provided and mixed with the sugar and the biological component. It is particularly preferred that the material is positively charged but in some alternative embodiments, the material may be negatively charged or may have no charge.

It is preferred that the sugar forms an amorphous solid matrix.

Preferably, the charged material has a pI value of higher than 7 and a positive charge of at least 0. In some embodiments, it has a positive charge of 0 to 5.

Conveniently, the charged material has a pI value higher than 10 with a positive charge of at least +5.

Preferably, the sugar is a disaccharide, a trisaccharide an oligosaccharide or a mixture thereof.

Advantageously, the sugar comprises sucrose, trehalose, umbelliferose, raffinose, stachyose, verbascose, melezitose, or mixtures thereof.

Conveniently, the sugar comprises sucrose and raffinose.

Alternatively, the sugar comprises trehalose and raffinose.

Alternatively, the sugar comprises trehalose and stachyose.

Alternatively, the sugar comprises sucrose and stachyose.

Preferably, the sugar comprises between 80% and 90% sucrose and 10% and 20% raffinose, more preferably 85% sucrose and 15% raffinose.

Alternatively, the sugar comprises between 80% and 90% trehalose and 10% and 20% raffinose, more preferably 85% trehalose and 15% raffinose.

Alternatively, the sugar comprises between 70% and 80% by volume sucrose and between 20% and 30% by volume stachyose, preferably 75% by volume sucrose and 25% by volume of stachyose.

Alternatively, the sugar comprises between 70% and 80% by volume trehalose and between 20% and 30% by volume stachyose, preferably 75% by volume trehalose and 25% by volume of stachyose.

Advantageously, the sugar comprises a mixture of a disaccharide with trisaccharide or a tetrasaccharide.

Conveniently, an extract of a plant seed or analogue thereof is also provided, the extract being capable of effecting desiccation tolerance of biological components.

Preferably, the positively charged material comprises a late embryogenesis abundant protein, a histone protein or a high mobility group protein.

Advantageously, the histone protein is histone 2A.

Conveniently, the sugar comprises an oligosaccharide and sucrose and/or umbelliferose and/or trehalose.

Preferably, the ratio of sucrose or umbelliferose or trehalose to oligosaccharide is between 0.9 and 1.1, preferably 1.0.

Advantageously, the ratio of sucrose or umbelliferose or trehalose to oligosaccharide is less than 1.0.

Conveniently, the biological component comprises an enzyme, a cell growth supplement, a vaccine preparation a cell, a platelet, a virus, an antibody or an antibody fragment, a pharmaceutical, an antibiotic, peptide, protein, nucleotide, nucleoside or polynucleic acid.

It is particularly preferred that the biological component comprises a virus, enzyme or protein.

Advantageously, the virus is a bacteriophage.

Alternatively, the virus is a DNA or an RNA virus.

Conveniently, the virus is measles virus, polio virus, rotavirus, human papiloma virus, respiratory syncitial virus, HIV, influenza virus, Dengue virus, Hepatitis virus, Yellow Fever virus, Varicella virus, Diptheria virus, Mumps virus, Rubella virus, or Japanese encephalitis virus.

Preferably, the biological component is an isolated biological component.

Advantageously, the biological component has been isolated from blood, milk, urine or cell-culture media.

Alternatively, the biological component is isolated from a virus, a prokaryotic cell, eukaryotic cell, plant or fungal source.

In some embodiments, the biological component is not a cell or is not a platelet.

According to a further aspect of the present invention, there is provided a desiccated or preserved product of the invention for use in medicine.

It is preferred that the method of preserving a biological component comprises the step of: (i) mixing the biological component with a sugar and a positively charged protein; and (ii) converting the sugar into an amorphous solid matrix. However, this is not essential to the invention.

Conveniently, the method comprises the step of drying the mixture, preferably by freeze drying.

Advantageously, the method further comprises subjecting the mixture to a vacuum.

Conveniently, the vacuum is applied at a pressure of 200 mbar or less, preferably 100 mbar or less.

Advantageously, the vacuum is applied for a period of at least 10 hours, preferably 16 hours or more.

Conveniently, step (ii) of the method comprises freezing the mixture, preferably by snap freezing.

Preferably, the method comprises the step of freezing the mixture at a temperature of −30° C. or less, preferably −78° C. or less, more preferably −196° C. or less.

Preferably the method further comprises the step of recovering the biological component by dissolving the dried mixture in a medium.

Conveniently, the method further comprises the step of processing the mixture into a formulation suitable for administration as a dry powder injection.

Advantageously, the method further comprises the step of processing the mixture into a formulation suitable for administration as a liquid injection.

Preferably, the method further comprises the step of processing the mixture into a formulation suitable for administration via ingestion or via the pulmonary route.

Conveniently, the step of drying is carried out by osmosis.

Alternatively, the step of drying comprises osmosis followed by lyophilisation.

Alternatively, the step of drying comprises osmosis followed by vacuum desiccation.

Advantageously, the method further comprises the step of storing the mixture at a temperature of at least 0° C., preferably at least 4° C., more preferably at least 10° C., more preferably at least 20° C. and more preferably at least 25° C. for a period of at least 24 hours, preferably at least 7 days.

In some embodiments, the biological component comprises a mixture of biological components.

It is preferred that in embodiments where the biological component comprises a cell, the method further comprises the step of rehydrating the cell and growing the cell.

Advantageously, mixing the sugar with the biological component produces a water-soluble vitreous matrix.

According to yet another aspect of the present invention there is provided a pharmaceutical composition comprising a desiccated or preserved product of the invention and a pharmaceutically acceptable carrier, excipient or diluent.

In this specification, the term “biological component” means any molecule, compound or structure (including, for example, viruses, cells and tissues) which is obtainable from a living source or is itself living.

In this specification, the term “virus” includes both “wild type viruses” and mutant viruses such as the attenuated viruses which form some vaccines.

In this specification, the terms “vitreous” or “vitreous-glass” are used in the general sense, to signify an amorphous non-crystalline solid (or semi-solid) rather than implying or involving any process of vitrification. In contrast, vitrification involves drying at elevated temperatures and subsequent cooling as outlined in WO99/27071.

Furthermore, the term “amorphous” means non-structured and having no observable regular or repeated organisation of molecules (i.e. non-crystalline).

In this specification, the term “snap freezing” involves submersion in liquid nitrogen at −196° C. until the solution is rendered solid.

Whilst not wishing to be bound by theory, it is believed that the present invention works as a result of the interplay between the charged material(s), the biological component to be protected and the amorphous non-crystalline solid support. More specifically, the charged material interacts hydrostatically with the biological components, displacing water of hydration. Upon drying, this intimate interaction between the charged material and the biological component is maintained with the help of the solidifying support structure generated by the sugar molecule. The solidified sugar's main role is in providing a support matrix for the charged material (e.g. histone 2a) affording the majority of stabilisation in concert with the sensitive biological component (e.g. a virus).

### DETAILED DESCRIPTION

In embodiments of the present invention there are provided sugars and other compounds present in plant seeds (or substances sharing their physical attributes e.g. histone proteins) which are capable of being reduced to a vitrified glass (that is to say, an amorphous, i.e. non-crystalline, matrix) by the removal of water of hydration and which offer desiccation and thermal protection to sensitive biological compounds e.g. virus particles, when temperature increases either in the form of a sugar-glass or protein-sugar-glass.

In one embodiment of the invention, sugars and other compounds commonly found in mature seeds (or physically homologous substances e.g. histone 2A (Kossel, A. (1928) The Protamines and Histones)) are combined and mixed with the substance to be protected prior to desiccation in the form of a vitreous glass using methods known in the art e.g. lyophilisation.

In another embodiment of the invention, additional components which contribute to the desiccation tolerance of seeds e.g. LEAs or analogous compounds from other sources e.g. mammalian origin such as histone proteins, are included prior to desiccation in the form of vitreous protein-sugar glass to enhance the desiccation or thermal tolerance of the biological compound to be protected. Histone proteins are common mammalian proteins possessing several physical properties analogous with LEAs.

Other examples of suitable positively charged proteins includes Histone 2B, Histone 3, Histone 4 and other DNA binding proteins. In other embodiments, the positively charged protein is a high mobility group protein, that is to say a non-histone protein involved in chromatin structure or gene regulation.

The material to be conferred with desiccation or thermal tolerance is isolated from a natural source in some embodiments, including viral, prokaryotic cells, eukaryotic cells, plant or fungal.

Alternatively, in some embodiments, the material to be protected is a synthesised compound such as a pharmaceutical e.g. antibiotics or peptides, proteins, nucleotides, nucleosides or polynucleic acids.

In some embodiments several compounds are preserved together, either mixed prior to processing in the preserving matrix or subsequently.

In preferred embodiments, the product is preserved by a method comprising snap freezing and then drying the product. Snap freezing is achieved by, for example, immersing the product in liquid nitrogen or dry ice.

In some embodiments of the invention, the product is dried, for example after freezing. In certain embodiments, drying is carried out using vacuum desiccation at around 10 Torr. However vacuum desiccation is not essential to the invention and in other embodiments, the product is spun (i.e. rotary desiccation) lyophilised (as is described further below) or boiled.

The compounds may be lyophilised, either in a range of containers including ampoules and vials, or directly onto plastic for subsequent rehydration for use.

In another embodiment of the invention, viable cells are rendered desiccation tolerant for subsequent growth following rehydration and growth in suitable media.

The composition may be formed by drying using any of the range of processes known in the art but preferably lyophilisation.

The composition once formed may be further processed e.g. milled to form a fine powder suitable for pulmonary administration, or for powder injection, or reconstituted in a suitable medium for injection.

In some embodiments, the products of the invention are administered to individuals in a method of treatment or prophylaxis. In some embodiments, the products of the invention comprise part of a pharmaceutical composition which also comprises a pharmaceutically acceptable carrier, diluent or excipient (see Remington's Pharmaceutical Sciences in US Pharmacopoeia, 1984, Mack Publishing Company, Easton, Pa., USA). The dose required for a patient may be determined using methods known in the art, for example, by dose-response experiments. A particular example where the present invention may be used in a method of treatment or prophylaxis is in the administration of a live vaccine to a patient in need of vaccination. The product of the invention may be administered by a range of routes, for example, orally or parenterally.

### EXAMPLES

### Example 1

An 85% w/v solution of sucrose and 15% w/v raffinose in phosphate buffered saline (PBS), was mixed with an equal volume of purified recombinant adenovirus (6.8×10¹² pfu/ml) expressing the reporter gene β-galactosidase and 10% w/v bovine serum albumin (BSA). The mixture was aliquoted into 100 μl aliquots and freeze dried by first freezing in liquid N₂ and then subjected to a vacuum of 100 mbar for 16 hours. Samples were then either immediately placed at −70° C. until required, or heated at 65° C. for 7 or 14 days. The results are shown in Table 1.

TABLE 1

| Days stored at | | Titre (pfu/ml) average of 3 |
|---|---|---|
| 65° C. | Protection | estimations |
| | Control (titre of viral stock) | 5.8 × 10¹² |
| 0 | Protected | 2.7 × 10¹⁰ |
| 0 | Control (no protection) | 3.0 × 10¹² |
| 7 | Protected | 2.8 × 10⁹ |
| 7 | Control (no protection) | 0 |
| 14 | Protected | 1.2 × 10¹⁰ |
| 14 | Control (no protection) | 0 |

### Example 2

According to the process described in Example 1, further samples of preserved virus were prepared, and following lyophilisation samples were either immediately frozen, or heated at 95° C. for 3 or 7 days. The results are shown in Table 2.

TABLE 2

| | | Titre (pfu/ml) average of |
|---|---|---|
| Days stored at 95° C. | Protection | 3 estimations |
| 0 | Protected | 7.1 × 10¹² |
| 0 | Control (no protection) | 4.2 × 10¹² |
| 3 | Protected | 8.1 × 10⁹ |
| 3 | Control (no protection) | 0 |
| 7 | Protected | 9.0 × 10⁹ |
| 7 | Control (no protection) | 0 |

### Example 3

A 293 cell monolayer was inoculated with a recombinant adenovirus expressing the reporter gene EGFP. When full cytopathic effect was evident, cells were collected by scraping and lysed by sonication. The lysed cells were then mixed with cell supernatant to form the viral stock. An 85% w/v solution of sucrose and 15% w/v raffinose in phosphate buffered saline (PBS), was mixed with an equal volume of recombinant adenovirus (5×10⁶ pfu/ml) and 10% w/v bovine serum albumin (BSA). The mixture was aliquoted into 100 μl aliquots and freeze dried by first freezing in liquid N₂ and then subjected to a vacuum of 100 mbar for 16 hours. Samples were then either immediately placed at −70° C. until required, or heated at 65° C. for 7 or 14 days. The results are shown in Table 3.

TABLE 3

| Days stored at | | Titre (pfu/ml) average of |
|---|---|---|
| 65° C. | Protection | 3 estimations |
| | Control (titre of viral stock) | 4.3 × 10⁶ |
| 0 | Protected | 3.7 × 10⁴ |
| 0 | Control (no protection) | 3.1 × 10⁶ |
| 3 | Protected | 7.8 × 10³ |
| 3 | Control (no protection) | 0 |
| 7 | Protected | 1.2 × 10⁴ |
| 7 | Control (no protection) | 0 |

### Example 4

A solution was prepared comprising 3 volumes of a 1 g/ml sucrose (in phosphate buffered saline), 1 volume of 1 g/ml Stachyose (in phosphate buffered saline), and 1 volume of 1 mg/ml Histone 2A (Boehringer Mannheim) (in phosphate buffered saline). The solution was aliquoted into 250 μl volumes and 50 μl of recombinant adenovirus (5×10⁶ pfu/ml) was added. After mixing, samples were either stored at −70° C. until needed or freeze dried by first freezing in liquid N₂ and then subjected to a vacuum of 100 mbar for 16 hours. After freeze drying, samples were then either placed at −70° C. until required, or heated at 65° C. for 7 days. The results are shown in Table 4.

TABLE 4

| | | Titre (pfu/ml) average of |
|---|---|---|
| Days stored at 65° C. | Protection | 3 estimations |
| 0 | Protected | 3.2 × 10⁵ |
| 0 | Control (no protection) | 3.1 × 10⁵ |
| 7 | Protected | 4.1 × 10⁵ |
| 7 | Control (no protection) | 0 |

### Example 5

A solution was prepared comprising 3 volumes of a 1 g/ml sucrose (in phosphate buffered saline), 1 volume of 1 g/ml Stachyose (in phosphate buffered saline), and 1 volume of 1 mg/ml Histone 2A (Boehringer Mannheim) (in phosphate buffered saline). The solution was aliquoted into 50 μl volumes and 5 μl of 1 mg/ml β-Galactosidase After mixing, samples were either stored at −70° C. until needed or freeze dried by first freezing in liquid N₂ and then subjected to a vacuum of 100 mbar for 16 hours. After freeze drying, samples were then either placed at −70° C. until required, or heated at 45° C. for various times as indicated. The results are shown in Table 5.

TABLE 5

| | | % Enzyme function |
|---|---|---|
| Time at 45° C. | Protection | remaining (ave of 3 est) |
| 5 min | Protected | 45 |
| 20 min | Protected | 45 |
| 30 min | Protected | 46 |
| 40 min | Protected | 43 |
| 5 min | Control Unprotected | 0.0017 |

### Example 6

A solution was prepared comprising 3 volumes of a 1 g/ml sucrose (in phosphate buffered saline), 1 volume of 1 g/ml Stachyose (in phosphate buffered saline), and 1 volume of 1 mg/ml Histone 2A (Boehringer Mannheim) (in phosphate buffered saline). The solution was aliquoted into 50 μl volumes and 5 μl of 1 mg/ml Photinus Pyralis Luciferase. After mixing, samples were either stored at −70° C. until needed or freeze dried by first freezing in liquid N₂ and then subjected to a vacuum of 100 mbar for 16 hours. After freeze drying, samples were then either placed at −70° C. until required, or heated at 65° C. for various times as indicated. The results are shown in Table 6.

TABLE 6

| | | % |
|---|---|---|
| | | Enzyme function |
| | | remaining |
| Time at Temperature | Protection | (ave of 3 est) |
| 2 hour @ 65° C. | Protected | 83 |
| 4 hour @ 65° C. | Protected | 55 |
| 24 hour @ 65° C. | Protected | 29 |
| 96 hour @ 65° C. | Protected | 5 |
| 120 hour @ 65° C. | Protected | 5 |
| 120 hour @ 26° C. | Protected | 74 |
| 120 hour @ 26° C. then 2 hr @ 65° C. | Protected | 84 |
| 120 hour @ 26° C. then 4 hr @ 65° C. | Protected | 56 |
| 15 min @ 65° C. | Unprotected | 0 |
| 45 min @ 65° C. | Unprotected | 0 |

### Example 7

### Poliovirus

Poliovirus: Sabin Strain Poliovirus Type 1. (titre log₁₀ CClD₅₀=8.0) was mixed (1:5 v/v) with excipient (at a ratio of 3:1:1 v/v comprising sucrose (100% w/v); stachyose (100% w/v); Histone H2A (1 mg/ml) in PBSA respectively). The mixture was dried by lyophilisation at a temperature of −30° C. for 2 days. After this time samples were stored until use at −70° C. or used immediately. Poliovirus was assayed using a CClD₅₀ methodology in Hep 2C cells. The results are shown in Table 7.

TABLE 7

| Treatment | Log₁₀ CCID₅₀/ml |
|---|---|
| Pre-lyophilisation | 7.3 |
| Post-lyophilisation | 5.7 |
| Untreated (post-lyophilisation) | 5.8 |
| Treated 37° C. 7days | 2.3 |

### Example 8

### Measles Virus

Measles virus, Schwarz strain with a titre of 5.45 log₁₀ pfu/ml, was mixed (1:5 v/v) with excipient (at a ratio of 3:1:1 v/v comprising sucrose (100% w/v); stachyose (100% w/v); Histone H2A (1 mg/ml) in PBSA respectively). The mixture was dried by lyophilisation at −30° C. for 2 days. After this time samples were stored until use at −70° C. or used immediately. Measles was assayed using a plaque assay in VERO cells. The results are shown in Table 8.

TABLE 8

| Treatment | Titre (pfu/ml) |
|---|---|
| Pre-lyophilisation | 9.3 × 10⁴ |
| Post-lyophilisation | 8.1 × 10⁴ |
| Untreated (post-lyophilisation) | 6 × 10⁴ |
| Treated 37° C. 7days | 5 × 10⁴ |

### Example 9

Measles virus, Schwarz strain with a titre of 5.45 log₁₀ pfu/ml., was mixed (1:5 v/v) with excipient (at a ratio of 3:1:1 v/v comprising sucrose (100% w/v); stachyose (100% w/v); Histone H2A (1 mg/ml) in PBSA, respectively). The mixture was dried by Vacuum desiccation whereby samples were dried at room temperature for 17 hours. After this time samples were stored until use at −70° C. or used immediately. Measles was assayed using a plaque assay in VERO cells. The results are shown in Table 9.

TABLE 9

| Treatment | Titre (pfu/ml) |
|---|---|
| Pre-lyophilisation | 6 × 10⁴ |
| Post-lyophilisation | 5.9 × 10⁴ |
| Untreated (post-lyophilisation) | 6.6 × 10⁴ |
| Treated 37° C. 7days | 5 × 10⁴ |

### REFERENCES

Galau, G. A., D. W. Hughes, and L. Dure. 1986. Abscisic acid induction of cloned cotton late embryogenesis-abundant (lea) mRNAs. Plant Molecular Biology 7: 157-170.
Bewley, J. D. and M. J. Oliver. 1992. Desiccation tolerance in vegetative plant tissues and seeds: protein synthesis in relation to desiccation and a potential role for protection and repair mechanisms. In Osmond, C. B.; Somero, G. (ed.), Water and life: a comparative analysis of water relationships at the organic, cellular and molecular levels. Springer Verlag, Berlin.
Kermode, A. R. 1997. Approaches to elucidate the basis of desiccation tolerance in seeds. Seed Science Research 7: 75-95.
Koster, K. L. and A. C. Leopold. 1988. Sugars and desiccation tolerance in seeds. Plant Physiology 88: 829-832.
Leprince, O., R. Bronchart, and R. Deltour. 1990. Changes in starch and soluble sugars in relation to the acquisition of desiccation tolerance during maturation of Brassica campestris seeds. Plant Cell and Environment 13: 539-546.
Blackman, S. A., R. L. Obendorf, and A. C. Leopold. 1992. Maturation proteins and sugars in desiccation tolerance of developing soybean seeds. Plant Physiology. 100: 225-230.
Horbowicz, M. and R. L. Obendorf. 1994. Seed desiccation tolerance and storability: Dependence on flatulence-producing oligosaccharides and cyclitols—review and survey. Seed Science Research 4: 385-405.
Obendorf, R. L. 1997. Oligosaccharides and galactosyl cyclitols in seed desiccation tolerance. Seed Science Research 7: 63-74.
Leopold, A. C. and C. W. Vertucci. 1986. Physical attributes of desiccated seeds. pp. 22-34 in Leopold, A. C. (Ed.) Membranes, metabolism and dry organisms. Ithaca, London, Comstock.
Crowe, J. H., L. M. Crowe, J. F. Carpenter, and C. A. Wistrom. 1987. Stabilization of dry phospholipid bilayers and proteins by sugars. Biochemical Journal 242: 1-10.
Crowe, J. H., F. A. Hoekstra, and L. M. Crowe. 1992. Anhydrobiosis. Annual Review of Physiology 54: 579-599.
Hoekstra, F. A. and T. van Roekel. 1988. Desiccation tolerance of Papaver dubium L. pollen during its development in the anther: possible role of phospholipid and sucrose content. Plant Physiology 88: 626-632.
Hoekstra, F. A., J. H. Crowe, and L. M. Crowe. 1991. Effect of sucrose on phase behavior of membranes in intact pollen of Typha latifolia L., as measured with Fourier transform infrared spectroscopy. Plant Physiology 97:1073-1079.
Clegg, J. S. 1986. The physical properties and metabolic status of Artemia cysts at low water content: the ‘Water Replacement Hypothesis’. pp. 169-187 in Leopold, A. C. (Ed.) Membranes, metabolism and dry organisms. Ithaca, N.Y., Cornell University Press.
Williams, R. J. and A. C. Leopold. 1989. The glassy state in corn embryos. Plant Physiology 89: 977-981.
Koster, K. L. 1991. Glass formation and desiccation tolerance in seeds. Plant Physiology 96: 302-304.
Bruni F, Careri G, Leopold A C. Critical exponents of protonic percolation in maize seeds. Phys Rev A. 1989 Sep. 1; 40(5):2803-2805.
Leopold, A. C., W. Q. Sun, and I. Bernal-Lugo. 1994. The glassy state in seeds: analysis and function. Seed Science Research 4: 267-274.
Galau, G. A., D. W. Hughes, and L. Dure. 1986. Abscisic acid induction of cloned cotton late embryogenesis-abundant (lea) mRNAs. Plant Molecular Biology 7: 157-170.
Koster K L, Webb M S, Bryant G, Lynch D V. Interactions between soluble sugars and POPC (1-palmitoyl-2-oleoylphosphatidylcholine) during dehydration: vitrification of sugars alters the phase behavior of the phospholipid. Biochim Biophys Acta. 1994 Jul. 13; 1193(1):143-50.
Galau G A, Bijaisoradat N, Hughes D W. Accumulation kinetics of cotton late embryogenesis-abundant mRNAs and storage protein mRNAs: coordinate regulation during embryogenesis and the role of abscisic acid. Dev Biol. 1987 September; 123(1):198-212.
Bruni F, Careri G, Leopold A C. Critical exponents of protonic percolation in maize seeds. Phys Rev A. 1989 Sep. 1; 40(5):2803-2805.
Wolkers W F, Hoekstra F A. Aging of Dry Desiccation-Tolerant Pollen Does Not Affect Protein Secondary Structure. Plant Physiol. 1995 November; 109(3):907-915.

1-81. (canceled)

82. A desiccated or preserved product comprising a positively charged material which is a protein and has a pI value of higher than 7, a biological component and a sugar in the form of an amorphous solid matrix.

83. A desiccated or preserved product according to claim 82 wherein the charged material has a pI value of higher than 10 with a positive charge of at least +5.

84. A desiccated or preserved product according to claim 82 wherein the charged material comprises a late embryogenesis abundant protein, histone protein or a high mobility group protein.

85. A desiccated or preserved product according to claim 84 wherein the histone protein is histone 2A.

86. A desiccated or preserved product according to claim 82 wherein the sugar
(a) is a disaccharide, a trisaccharide, an oligosaccharide or a mixture thereof, or
(b) comprises sucrose, trehalose, umbelliferose, raffinose, stachyose, verbascose, melezitose, or mixtures thereof.

87. A desiccated or preserved product according to claim 82 further comprising an extract of a plant seed or analogue thereof, the extract being capable of effecting desiccation tolerance of biological components.

88. A desiccated or preserved product according claim 82 wherein the biological component comprises an enzyme, a cell growth supplement, a vaccine preparation, a cell, a platelet, a virus, an antibody or an antibody fragment, a pharmaceutical, an antibiotic, peptide, protein, nucleotide, nucleoside or polynucleic acid.

89. A desiccated or preserved product according to claim 88 wherein the virus is
(a) a bacteriophage;
(b) a DNA or an RNA virus; or
(c) measles virus, polio virus, rotavirus, human papiloma virus, respiratory syncitial virus, HIV, influenza virus, Dengue virus, Hepatitis virus, Yellow Fever virus, Varicella virus, Diptheria virus, Mumps virus, Rubella virus, or Japanese encephalitis virus.

90. A desiccated or preserved product according to claim 88 wherein the biological component has been isolated from
(a) blood, milk, urine or cell-culture media; or
(b) a virus, a prokaryotic cell, eukaryotic cell, plant or fungal source.

91. A method of preserving a biological component comprising the steps of: (i) mixing the biological component with a sugar and a positively charged material which is a protein and has a pI value of higher than 7; and (ii) converting the sugar into an amorphous solid matrix.

92. A method according to claim 91 wherein
(a) the charged material is as defined in any one of claims 83 to 85;
(b) the sugar is as defined in claim 86; and/or
(c) the biological component is as defined in any one of claims 88 to 90.

93. A method according to claim 91 wherein step (ii) comprises freeze-drying the mixture.

94. A method according to claim 91 further comprising mixing the biological component with an extract of a plant seed or analogue thereof, the extract being capable of effecting desiccation tolerance of biological components.
